(19) United States
(12) Patent Application Publication
(10) Pub. No.: US 2008/0183064 A1
(43) Pub. Date: Jul. 31, 2008

(54) Multi-sensor distortion detection method and system

(75) Inventors: Jason Rene Chandonnet, Lowell, MA (US); Vernon Thomas Jensen, Draper, UT (US); Jon Thomas Lea, Hampstead, NH (US)

Correspondence Address: GE HEALTHCARE;c/o FLETCHER YODER, PC, P.O. BOX 692289, HOUSTON, TX, 77269-2289, US

(73) Assignee: General Electric Company

(21) Appl. No.: 11/699,862

(22) Filed: Jan. 30, 2007

(51) Int. Cl. A61B 5/00; G01B 7/00

(52) U.S. Cl. 600/407; 324/207.12

(57) ABSTRACT

A method for detecting EM field distorting includes sampling a sensor assembly positioned within a volume of interest to acquire measurements of EM fields within the volume of interest, and monitoring the measurements to detect EM field distortion within the volume of interest. The sensor assembly includes a set of EM transmitters and a set of EM receivers fixed thereon. A system for detecting EM field distorting includes a sensor assembly for positioning within a volume of interest, wherein the EM sensor assembly comprises a set of EM receivers, and a set of EM transmitters, wherein the EM receivers and the EM transmitters are disposed at fixed locations on the sensor assembly. The system further includes a tracker configured to sample the sensor assembly to acquire measurements of EM fields generated by the EM transmitters, and monitor the measurements to detect EM field distortion within the volume of interest.

### BACKGROUND

This disclosure relates generally to tracking systems that use magnetic fields, such as for surgical interventions and other medical procedures. More particularly, this disclosure relates to an apparatus and method for detecting magnetic field distortion in such systems.

Tracking systems have been used to provide an operator (e.g., a physician) with information to assist in the precise and rapid positioning of a medical (e.g., surgical) device in a patient's body. In general, an image is displayed for the operator that includes a visualization of the patient's anatomy with an icon or image representing the device superimposed thereon. As the device is positioned with respect to the patient's body, the displayed image is updated to reflect the correct device coordinates. The image of the patient's anatomy may be generated either prior to or during the medical or surgical procedure. Moreover, any suitable medical imaging technique, such as X-ray, computed tomography (CT), magnetic resonance imaging (MRI), positron emission tomography (PET), and ultrasound, may be utilized to provide the basic image in which the device tracking is displayed.

To determine device location, tracking systems have utilized electromagnetic (EM) fields. During these procedures, signals are transmitted from one or more EM transmitters to one or more EM receivers. In one example, an EM receiver is mounted in an operative end of the device. In general, the EM transmitters generate an electromagnetic field that is detected by the EM receivers and then processed to determine the device location, for example, the position and orientation, including the X, Y and Z coordinates and the roll, pitch and yaw angles.

However, as those of ordinary skill in the art appreciate, the presence of field distorting objects may result in distortions in the magnetic field emitted from the EM transmitters and thereby change the magnitude and direction of this field. For example, the presence of a signal from another source, magnetic fields of eddy current in conductive objects, or the field distorting effects of a ferro-magnetic object can result in these distortions. Unless compensated for, these distortions will result in error in the determined location of the device.

One source of magnetic field distortions may be the equipment utilized in the tracking system itself. For example, certain tracking systems include a fixture containing one or more EM sensors that are attached to an imaging system, such as to the C-arm of an X-ray fluoroscopy system. As those of ordinary skill in the art will appreciate, these imaging systems typically include conducting objects (e.g., the C-arm) that result in the above-described field distortions. To compensate for this known distortion, a distortion map is generally created for each tracking system during the factory calibration process. This distortion map is used by the tracking system to compensate for this known distorting effect during the medical procedure.

An exemplary technique for creating the distortion map for a tracking system that includes an X-ray fluoroscopy system containing a C-arm, involves use of a precision robot. An EM transmitter is attached to an arm of the robot and moved to numerous points in space within the navigated volume. At each point, signals from the EM transmitter are detected by one or more EM receivers and then processed to determine a measured location of the transmitter with respect to the receiver, which is rigidly fixed to the C-arm of the X-ray fluoroscopy system. Because a precision robot is used, the real world location of the transmitter at each sampled point in the navigated volume is known. Accordingly, the measured location of the device detected by the receivers is compared to the transmitter's known real location to generate the distortion map that is used by the tracking system. By way of example, the distortion map may cross-reference the measured transmitter location with the known real transmitter location. However, to generate a complete distortion map, the transmitter must be positioned at numerous points within the navigated volume. This process of collecting the needed data points is time consuming and resource intensive. Moreover, extra time may be required to allow for the robot arm to stabilize at each point, and extreme care must be used to ensure that the system is not disturbed during data acquisition.

In addition to the tracking system itself, field distorting objects also may be present in the clinical environment where the tracking system is used. However, the impact of these field distorting objects on the magnetic field in the clinical environment is generally not known, and the field distorting objects are frequently transient. Techniques for detecting distorting objects during medical procedures have been developed. One such technique utilizes two receiver coil assemblies rigidly mounted at a known fixed distance, wherein the locations of virtual points are monitored to detect uniform distortions in the area of the medical device. However, these techniques only detect field distortions in the immediate vicinity of the two coil assemblies and do not convey the extent of field distortions in the larger navigated volume.

Accordingly, there is a need for an improved technique for detecting and correcting for magnetic field distortion. Particularly, there is a need for a technique that detects magnetic field distortion in and around a tracking system so that this distortion can be accounted for in the clinical environment.

### BRIEF DESCRIPTION

The present technique provides a novel method and apparatus for detecting EM field distortion. In accordance with one embodiment of the present technique, a method is provided for detecting EM field distortion. The method includes sampling a sensor assembly positioned within a volume of interest to acquire measurements of EM fields within the volume of interest. In this embodiment, the sensor assembly comprises a set of EM transmitters for generating the EM fields and a set of EM receivers for measuring the electromagnetic fields, wherein the EM transmitters and EM receivers are disposed at fixed locations on the sensor assembly. The method further includes monitoring the measurements to detect EM field distortion within the volume of interest.

In accordance with another aspect, another method for detecting EM field distortion is provided. The method includes positioning a sensor assembly fixed in relation to a patient. In this embodiment, the sensor assembly comprises a set of EM transmitters and a set of EM receivers, wherein the EM transmitters and the EM receivers are disposed at fixed locations on the sensor assembly. The method further includes positioning a device within the patient, the device comprising an EM sensor for generating an EM field or for measuring an EM field. The method further includes tracking the position of the device with respect to the sensor assembly. The method further includes sampling the sensor assembly to obtain measurements from the set of EM receivers of EM fields generated by the set of EM transmitters. The method further includes monitoring the measurements to detect EM field distortion within the volume of interest.

In accordance with another aspect, a system for detecting EM field distortions is provided. The system includes a sensor assembly for positioning within a volume of interest. In this embodiment, the EM sensor assembly comprises a set of EM receivers, and a set of EM transmitters, wherein the EM receivers and the EM transmitters are disposed at fixed locations on the sensor assembly. The system further includes a tracker configured to sample the sensor assembly to acquire measurements of EM fields generated by the EM transmitters, and monitor the measurements to detect EM field distortion within the volume of interest.

### DRAWINGS

These and other features, aspects, and advantages will become better understood when the following detailed description is read with reference to the accompanying drawings in which like characters represent like parts throughout the drawings, wherein:

FIG. 1 is a schematic illustration of an exemplary system for detecting magnetic field distortion implementing certain aspects of the present technique;

FIG. 2 is a schematic representation of an exemplary sensor assembly in accordance with certain aspects of the present technique;

FIG. 3 is a schematic representation of an alternative sensor assembly in accordance with certain aspects of the present technique;

FIG. 4 is a schematic representation of an alternative sensor assembly configured for placement around the torso of a patient in accordance with certain aspects of the present technique;

FIG. 5 is a schematic representation of an alternative sensor assembly configured for placement around the head of a patient in accordance with certain aspects of the present technique;

FIG. 6 is a schematic representation of an alternative sensor assembly having an alternative arrangement of EM sensors thereon in accordance with certain aspects of the present technique;

FIG. 7 is a schematic representation of an alternative sensor assembly in accordance with certain aspects of the present technique;

FIG. 8 a schematic representation of another alternative sensor assembly in accordance with certain aspects of the present technique;

FIG. 9 is a cross-sectional, side view of the alternative sensor assembly of FIG. 8;

FIG. 10 a schematic representation of another alternative sensor assembly in accordance with certain aspects of the present technique;

FIG. 11 is a cross-sectional, side view of the alternative sensor assembly of FIG. 10;

FIG. 12 is a schematic illustrating a system for detecting EM field distortion during an EM tracking procedure; and

FIG. 13 is a schematic illustration of a system for detecting and/or characterizing EM field distortion in a clinical environment.

### DETAILED DESCRIPTION

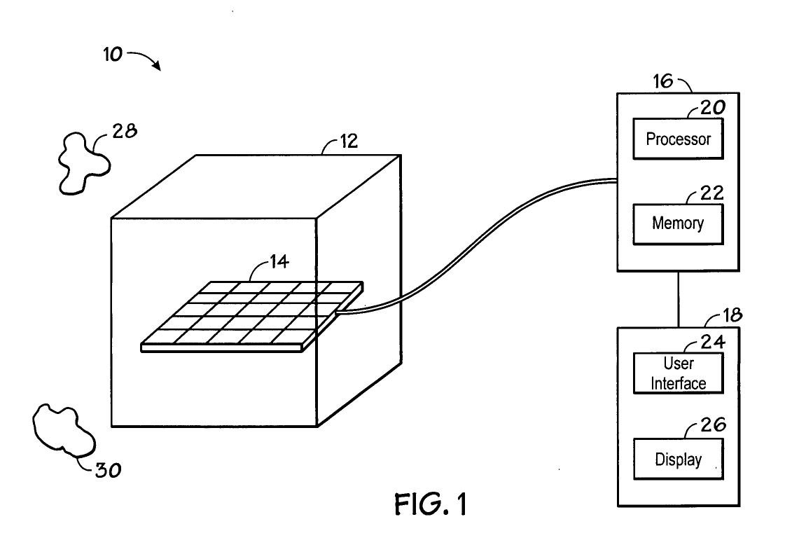

FIG. 1 illustrates diagrammatically a system 10 for detecting EM field distortion within a volume of interest 12. As illustrated, the system 10 generally includes an EM sensor assembly 14, a tracker 16, and an operator workstation 18.

In the illustrated embodiment, an operator positions EM sensor assembly 14 within the volume of interest 12 to detect EM field distortion therein. The volume of interest 12 may be any suitable volume where it is desired to detect and/or correct for magnetic field distortions. For example, the volume of interest 12 may be a volume to be navigated by a medical device, wherein a tracking system will be used to determine the location of the medical device in the volume of interest 12. EM sensor assembly 14 generally includes a set of EM receivers and a set of EM transmitters disposed on the sensor assembly at fixed locations with respect to each other. By way of example, the EM transmitters may be implemented as field generators with each sensor including three orthogonally disposed magnetic dipoles (e.g., current loops or electromagnets). In one embodiment, each of the EM transmitters and EM receivers may employ industry-standard coil architecture (“ISCA”). ISCA is defined as three approximately collocated, approximately orthogonal, and approximately dipole coils. In another embodiment, each EM transmitter may have a single dipole. Electromagnetic fields generated by each of the dipoles are distinguishable from one another by phase, frequency, time division multiplexing, and/or the like. As those of ordinary skill in the art will appreciate, the near-field characteristics of the electromagnetic fields may be used for coordinate determination. Other suitable techniques for using the EM transmitters for generating a field in which location detection may be achieved within the volume of interest 12 may be utilized with the present technique.

The EM receivers of the EM sensor assembly 14 may be configured to measure the electromagnetic field emitted by the EM transmitters. In one embodiment, the EM receivers may be configured in an ISCA having three dipoles. In another embodiment, the EM receivers may be configured having a single dipole. As will be appreciated, the mutual inductance of two EM sensors is the same, regardless of which is the receiver and the transmitter. Therefore, relative positioning and functionality of the EM receivers and EM transmitters on the EM sensor assembly 14 may be reversed.

The system 10 further includes tracker 16. In the illustrated embodiment, the field measurements from the EM receivers are output to the tracker 16 for processing. In one embodiment, the tracker 16 may monitor the field measurements from the EM receivers to determine an apparent location (e.g., position and/or orientation) of the each of the EM receivers with respect to the EM sensor assembly 14. In one embodiment, the tracker 16 may monitor the field measurements from the EM receivers to determine an apparent location of each of the EM transmitters with respect to the EM sensor assembly 14. Other aspects of the field measurements also may be monitored, for example, the gain of a single coil and/or the mutual inductance between an EM receiver and EM transmitter.

As previously mentioned, in some embodiments, each of the EM transmitters and/or EM receivers may be configured having a single coil. As will be appreciated, since a single coil is symmetrical about its roll axis, only five degrees of freedom (three of position and two of orientation) may be determined. Moreover, if two or more of the single coils have axes in different directions, all six degrees of freedom (three of position and three of orientation) for the set of two or more coils may be determined by tracker 16. Additionally, the gain of the single coil also may be monitored.

Moreover, in some embodiments, the EM receivers and/or EM transmitters may be configured in an ISCA having three dipole coils. Accordingly, the tracker 16 may determine the location (e.g., position and/or orientation) of the multiple coils of the ISCA individually or as a group. For example, tracker 16 can determine a position and/or orientation of each coil in the ISCA EM sensors, as well as the gain for each coil. Additionally, as the coils in ISCA point in different directions (have axes in different directions), tracker 16 can determine six degree of freedom for each coil in the ISCA EM sensors. Tracker 16 also can determine a position and orientation of the coils as a group to determine the six degrees of freedom for the ISCA EM sensor.

As will be appreciated, one or more computers may be used to implement tracker 16. In general, tracker 16, may include processor 20, which may include a digital signal processor, a CPU or the like, for processing the acquired signals. Tracker 16 further may include memory 22. It should be noted that any type of memory may be utilized in tracker 16. For example, memory 22 may be any suitable processor-readable media that is accessible by the tracker 16. Moreover, the memory 22 may be either volatile or non-volatile memory. Memory 22 may serve to save the tracking data as well as other system parameters. In addition, tracker 16 include may include electronic circuitry to provide the drive signals, electronic circuitry to receive the sensed signals, and electronic circuitry to condition the drive signals and the sensed signals. Further, the processor 20 may include processing to coordinate functions of the system 10, to implement navigation and visualization algorithms suitable for tracking and displaying the position and orientation of an instrument or device on a monitor. While in the illustrated embodiment, the tracker 16 is shown as being outside the EM sensor assembly 14 and/or operator workstation 18, in certain implementations, some or all of the tracker 16 may be provided as part of the EM sensor assembly 14 and/or the operator workstation 18.

As illustrated, operator workstation 18 includes user interface 24 and a display 26. User interface 24 may include a keyboard and/or mouse, as well as other devices such as printers or other peripherals. By way of example, the display 26 may be used to provide graphic feedback indicating areas within the volume of interest 12 that need additional data.

As those of ordinary skill in the art will appreciate, the presence of field distorting objects 28, 30 in or near the volume of interest 12 may result in distortion in the EM field generated by the EM sensor assembly 14. By way of example, the field distorting objects 28, 30 may be tables, fixtures, tools, electronic equipment, one or more components of an imaging system (e.g., a C-arm). One or more of these objects may be present in a clinical environment that would then distort EM fields used, for example, in EM device tracking.

As previously mentioned, a distortion map may be created during factory calibration to compensate for known distortions. However, this distortion map generally will not contain characterize the impact of distorting objects present in the volume of interest 12. However, the impact of these field distorting objects on the magnetic field in the clinical environment is generally not known, and the field distorting objects are frequently transient. For example, additional distorting objects (e.g., tools, tables) may be present in a clinical environment that were not accounted for during factory calibration. Techniques have been developed to detect field distortion in a clinical environment during a medical procedure. For example, one such technique utilizes two receiver coil assemblies rigidly mounted at a known fixed distance, wherein the locations of virtual points are monitored to detect uniform distortions in the area of the medical device. However, these techniques generally only detect field distortion in the immediate vicinity of the two coil assemblies and do not convey the extent of field distortion in the larger navigated volume.

Accordingly, the present technique allows for detecting field distortion caused by distorting objects in, and around, the volume of interest 12. As will be appreciated, the field distortions, such as those created by field distorting objects 28, 30 may be detected by monitoring EM field measurements acquired by sampling the EM sensor assembly 14. In one embodiment, an EM field error may be reported based on the monitored EM field measurements. This may be useful, for example, in a medical procedure where the location of a device (e.g., a catheter) positioned within the volume of interest 12 is tracked using tracker 16. In some embodiments, based on the detected field distortion, the compatibility of the volume of interest 12 for use with EM device tracking could be determined.

In addition, the present technique also allows for characterization of the field distortion within the volume of interest 12 based on the monitored EM field measurements. In one embodiment, tracker 16 could be calibrated based on the characterization of the field distortion. As in the case summarized above, this may be useful, for example, in a medical procedure where the location of a device (e.g., a catheter) positioned within the volume of interest 12 is tracked using tracker 16. Based on the calibration of the tracker 16, the tracked location of the device could be corrected to compensate for the detected field distortions. In one embodiment, a distortion map may be created that characterizes the field distortions detected in the volume of interest 12. In one embodiment, the distortion map may include a look-up table that, for example, cross-references the undistorted sensor locations with the distorted sensor locations.

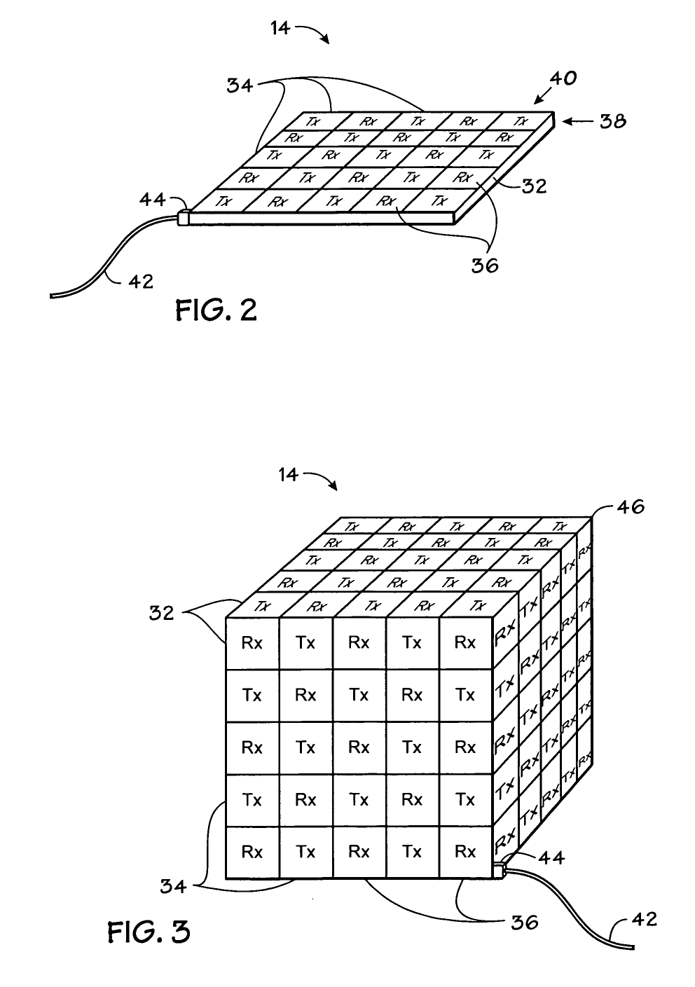

Referring now to FIG. 2, an EM sensor assembly 14 in accordance with one embodiment of the present technique is illustrated. In the illustrated embodiment, EM sensor assembly 14 comprises a sensor panel 32 that includes a set of EM transmitters 34 mounted on the sensor panel 32, and a set of EM receivers 36 mounted on the sensor panel 32. In general, the EM transmitters 34 and the EM receivers 36 are fixed on the sensor assembly 14 with respect to each other. In one embodiment, the sensor panel 32 is rigid so that the distance between the EM transmitters 34 and the EM receivers 36 is fixed. Alternatively, the EM transmitters 34 and EM receivers 36 may be mounted on the sensor panel 32 using any suitable technique. For example, to maintain the fixed distance, a rigid mount may be used to fix the EM transmitters 34 and EM receivers 36 to the sensor panel 32. While the sensor panel 32 is illustrated as having a generally rectangular shaped surface, those of ordinary skill in the art will appreciate the sensor panel 32 may have any suitable shape for positioning the EM transmitters 34 and EM receivers 36 thereon. For example, sensor panel 32 may have a generally circular or elliptical shaped surface.

As illustrated, the EM sensors (e.g., EM transmitters 34 and EM receivers 36) are positioned on the sensor panel 32 in a series of rows. In one embodiment, the rows of EM sensors are arranged on sensor panel 32 in an alternating arrangement along each row. For example, a row of EM sensors (denoted generally by reference number 38) alternates between an EM transmitter 34 and an EM receiver 36 along the row 38. As will be appreciated, transmitters and receivers in the series of rows may be arranged to form a corresponding series of columns. In one embodiment, the EM sensors are positioned in an alternating arrangement along each column, as well as in an alternating arrangement along each row. For example, a column of EM sensors (denoted generally by reference number 40) alternates between an EM transmitter 34 and an EM receiver 36 along the column 40. While the EM sensor assembly 14 illustrates EM transmitters 34 and EM receivers 36 arranged in an alternating manner, the present technique also encompasses other suitable sensor arrangements.

Because the sensors are disposed on the EM sensor assembly at fixed locations with respect to each other, the location of each of the EM transmitters 34 and each of the EM receivers 36 with respect to the EM sensor assembly 14 is known a priori. A variety of techniques may be used to determine the location of each EM transmitter 34 and each EM receiver 36 with respect to the EM sensor assembly 14. For example, the location (e.g., the position and orientation) of each of the sensors may be determined from engineering data based on the assembly of the EM sensor assembly 14. Alternatively, the location of each of the sensors may be determined during a factory calibration in an environment essentially free of field distortions. During this factory calibration, the sensor assembly 14 may be sampled and the monitored EM signals may be used to determine the location of each of the EM transmitters 34 and EM receivers 36 with respect to the EM sensor assembly 14.

Those of ordinary skill in the art will appreciate that the EM transmitters 34 and EM receivers 36 may be suitably spaced so as not to undesirably affect the sensing accuracy of a particular sensor with respect to its neighbors based on a variety of factors, including sensor size, range, and sensitivity. It should be noted that, while FIG. 2 illustrates uniform spacing between the EM transmitters 34 and EM receivers 36 on the sensor assembly 14, non-uniform spacing of the EM sensors is also encompassed by the present technique.

As will be appreciated, cable 42 is coupled to sensor assembly 14 and provides the necessary leads and/or wires for connection with the EM transmitters 34 and EM receivers 36 for proper operation of sensor assembly 14. Alternatively, the EM transmitters 34 and EM receivers 36 may be wireless. Moreover, sensor assembly 14 may comprise a variety of additional electronics 44, such as multiplexers, pre-amplifiers, analog-to-digital converters, or other digital signal processing components, coupled to the sensor panel 32.

While FIG. 2 illustrates a single sensor panel 32, sensor assembly 14 may include a plurality of sensor panels 32 arranged in two or more planes wherein each of the sensor panels 32 comprises one or more of the set of EM receivers 36 and one or more of the set of EM transmitters 34. By way of example, FIG. 3 illustrates a variation of sensor assembly 14 suitable for use with the present technique. In this variation, sensor assembly 14 comprises a box 46 that includes a sensor panel 32 on two or more sides. In the illustrated embodiment, box 46 is a cubic box that includes a sensor panel on each of five sides. As will be appreciated, the bottom of the five-box 46 may be open or closed. By way of example, the box 46 may have an open bottom where desired to have an open volume in sensor assembly 14. Moreover, while sensor assembly 14 is illustrated as including a cubic box, those of ordinary skill in the art will appreciate that, in certain embodiments, the sensor assembly 14 may be any suitable shape configured to allow placement of a plurality of sensor panels in two or more planes. For example, sensor assembly 14 may include a rectangular box or other suitable structure for placement of the sensor panels 32. Cable 42 connected to sensor assembly 14 provides the necessary leads and/or wires for connection with EM transmitters 34 and EM sensors 36 for proper operation of sensor assembly 14. Moreover, as illustrated on FIG. 3, sensor assembly 14 further comprises electronics 44 coupled to the box 46.

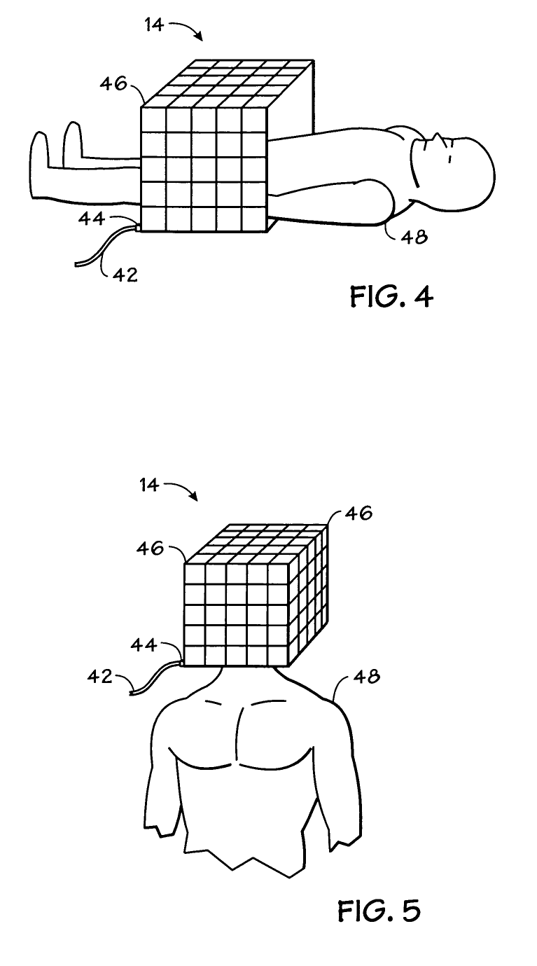

FIG. 4 illustrates a variation of sensor assembly 14 suitable for use with the present technique. In this variation, sensor assembly 14 comprises a box 46 having four sides and that includes a sensor panel 32 on each side. The remaining two sides of the box 46 are open to so that sensor assembly 14 has an opening therethrough. As illustrated by FIG. 4, the box 46 may be configured to be placed around a patient 48 in a desired location, such as the torso. Accordingly, the embodiment illustrated by FIG. 4 may be useful, for example, to detect and/or characterize field distortion in a clinical environment prior to or during EM device tracking. Placement around the torso of patient 48 may be desirable, for example, to track a device (e.g., a catheter) inserted into patient 48.

FIG. 5 illustrates another variation of sensor assembly 14 with an open bottom. In this variation, sensor assembly 14 comprises a box 46 having five sides and that includes a sensor panel 32 on each side. As illustrated by FIG. 5, the box 46 may configured to be placed around the head of a patient 48. As will be appreciated, the head and upper torso, including the ear, nose and throat area is constitutes one exemplary patient region where EM device tracking may be utilized. Accordingly, the embodiment illustrated by FIG. 5 may be useful, for example, to detect and/or characterize field distortion in a clinical environment prior to or during EM device tracking. When used during EM device tracking, the sensor assembly 14 placed around the head of patient 48 may be adapted to allow access to the head region.

FIG. 6 illustrates another variation of sensor assembly 14 suitable for use with the present technique. In this embodiment, sensor assembly 14 comprises a box 46 having a sensor panel 32 on two or more sides. Unlike the previously illustrated embodiments, the EM transmitters 34 and EM receivers 36 are not positioned on the sensor panel 32 in a series of rows that alternate between an EM transmitter and an EM receiver. Rather, in the illustrated embodiment, the EM receivers 36 are generally positioned in the center of each sensor panel 32, and the EM transmitters 34 are generally positioned in each corner of each EM sensor panel 32. As will be appreciated, the relative positioning and functionality of the EM receivers 36 and EM transmitters 34 on the EM sensor assembly 14 may be reversed.

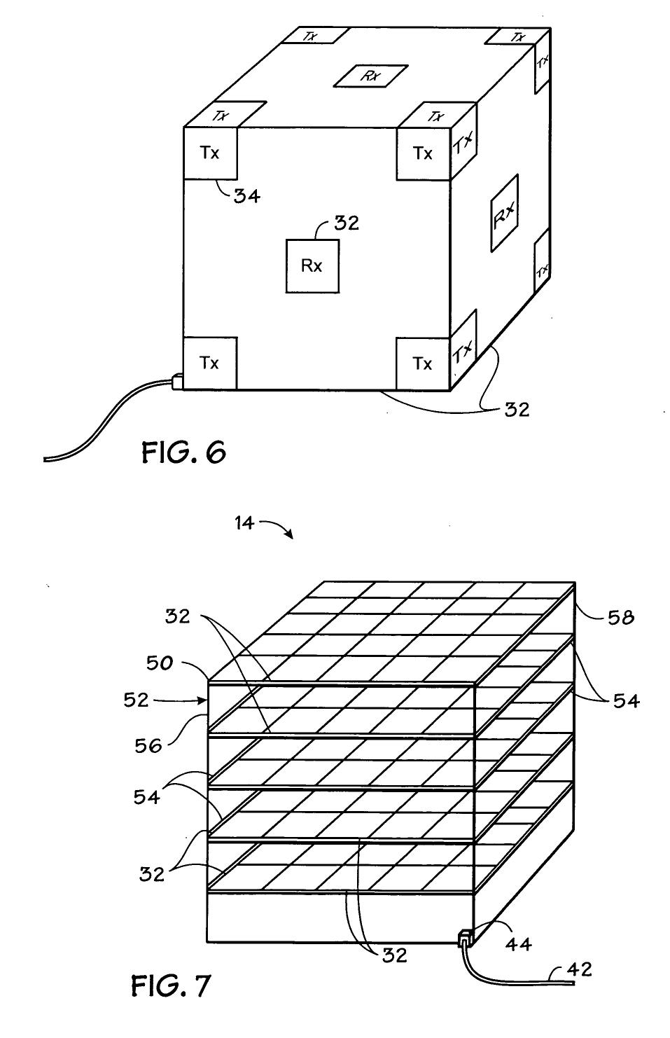

FIG. 7 illustrates another variation of sensor assembly 14 suitable for use with the present technique. Sensor assembly 14 comprises a rack system 50 made up of vertical support columns 52, and a plurality of horizontal rails 54 coupled to the vertical support columns 52. A plurality of sensor panels 32 are coupled to the horizontal rails 54 in a generally vertical arrangement along the vertical support columns 52. In the illustrated embodiment, vertical support columns 52 comprise at least one front vertical support column 56 and at least one rear vertical support column 58. In one embodiment, a pair of the horizontal rails 54 may be used to slidably mount a sensor panel 32 in the rack system 50. The sensor panels 32 may be coupled to the horizontal rails 54 by any of a variety of mechanisms, such as clips, screws, snaps or other suitable fasteners.

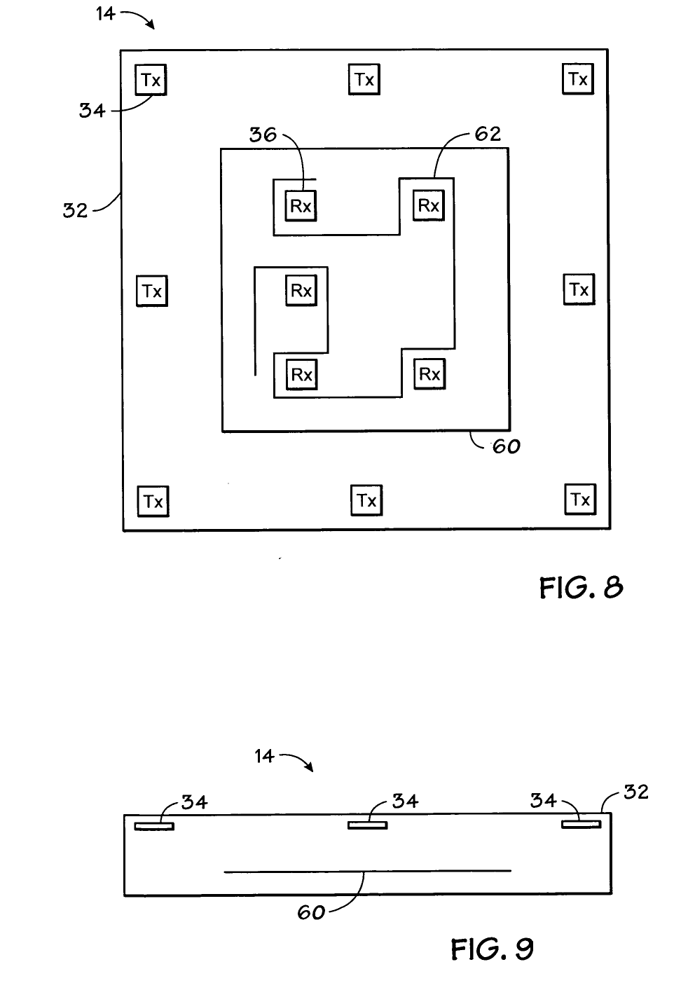

FIGS. 8 and 9 illustrate another variation of sensor assembly 14 suitable for use with the present technique. In the illustrated embodiment, sensor assembly 14 includes a set of EM transmitters 34 and a set of EM receivers 36 fixed on the sensor assembly 32 with respect to each other. In the embodiment illustrated, the sensor assembly 14 may include a printed circuit board 60. For example, a printed circuit board 60 may be comprises a set of EM receivers 36 printed thereon. In one embodiment, each of the EM receivers 36 printed on the printed circuit board 60 may have a single coil. In one embodiment, the EM transmitters 34 may be configured in an ISCA having three dipole coils while the EM receivers may be configured having a single coil. In some embodiments, EM transmitters 34 may also be printed on the printed circuit board 60. As illustrated, the EM transmitters 34 are arranged on the periphery of the sensor assembly 14. Moreover, in the illustrated embodiment, the EM transmitters 34 are on a different plane than the EM receivers 36. However, those of ordinary skill will appreciate that, in certain implementations, the EM receivers 36 may be on the same plane as the EM transmitters 34. In the illustrated embodiment, the printed circuit board 60 further includes a calibration coil 62 that transmits at a known frequency and current. Accordingly, the measured mutual inductance between the calibration coil 62 and the EM receivers 36 should generally be constant. Accordingly, tracker 16 may also monitor this mutual inductance. While not illustrated, sensor assembly 14 may further include additional electronics, such as multiplexers, pre-amplifiers, analog-to-digital converters, and additional digital processing equipment. As will be appreciated, the connection between the EM transmitters 34 and EM receivers 36 and tracker 16 may be wired or wireless.

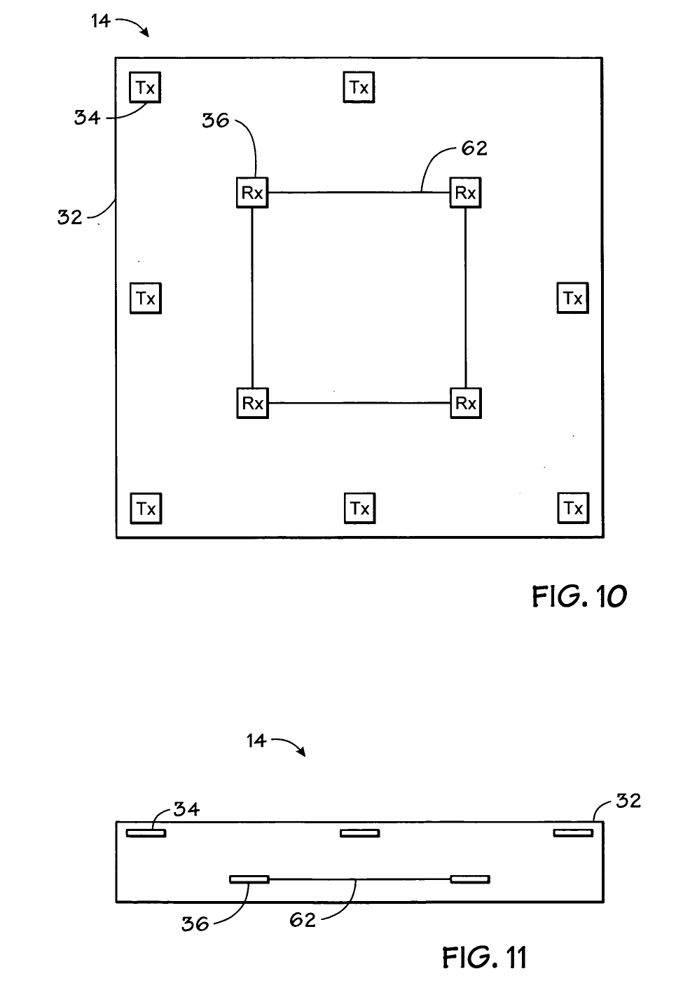

FIGS. 10 and 11 illustrate another variation of sensor assembly 14 suitable for use with the present technique. In the illustrated embodiment, sensor assembly 14 includes a set of EM transmitters 34 and a set of EM receivers 36 fixed on the sensor assembly 32 with respect to each other. In one embodiment, the EM transmitters 34 and the EM receivers 36 may be configured in an ISCA having three dipole coils. As illustrated, the EM transmitters 34 are arranged on the periphery of the set of EM receivers 36. Moreover, in the illustrated embodiment, the EM transmitters 34 are on a different plane than the EM receivers 36. However, those of ordinary skill will appreciate that, in certain implementations, the EM receivers 36 may be on the same plane as the EM transmitters 34. Sensor assembly 14 further includes a calibration coil 62 that transmits at a known frequency and current. Accordingly, the measured mutual inductance between the calibration coil 62 and the EM receivers 36 will ordinarily be constant. Accordingly, tracker 16 may also monitor this mutual inductance. While not illustrated, sensor assembly 14 may further include additional electronics, such as multiplexers, pre-amplifiers, analog-to-digital converters, and additional digital processing equipment. As will be appreciated, the connection between the EM transmitters 34 and EM receivers 36, and tracker 16 may be wired or wireless.

Those of ordinary skill in the art will appreciate that the EM sensor assembly 14 may be any suitable size for a particular application. By way of example, a typical tracking volume may have a cubic shape with a length, width, and height of up to about 2 feet (approximately 60 cm) in length. Accordingly, in some embodiments, the EM sensor assembly 14 may be sized to fill the desired tracking volume so that EM field distortions in the tracking volume, such as volume of interest 12, may be detected and/or characterized. In some embodiments, the EM sensor assembly 14 may be sized for placement on the head of a subject, such as in the embodiment illustrated in FIG. 5. For example, the EM sensor assembly 14 may be a five-sided box with an open bottom and having a length, width, and height in the range of from about 12 inches (approximately 30 cm) to about 18 inches (approximately 45 cm). In some embodiments, the EM sensor assembly 14 may be sized for placement around the torso of a patient, such as in the embodiment illustrated in FIG. 4. For example, the EM sensor assembly 14 may be a four sided box having a length, width, and height in the range of from about 18 inches (approximately 45 cm) to about 24 inches (approximately 60 cm). However, it should be recognized that the previously described sizes are merely exemplary and that a wide variety of sensor assemblies are encompassed within the present technique.

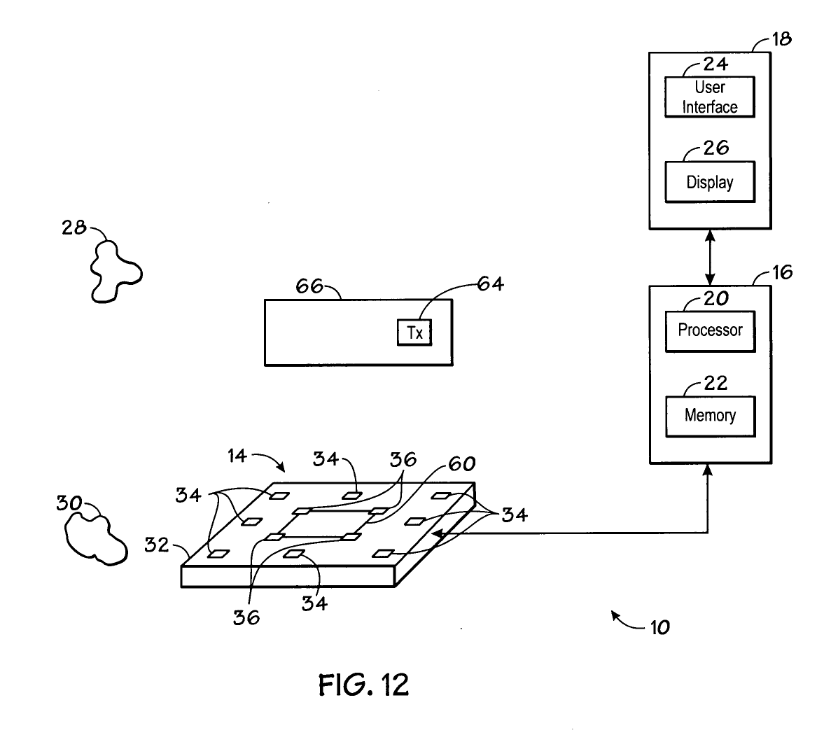

In one embodiment, the present technique may be used to detect magnetic field distortion during an EM tracking procedure, as previously mentioned. Referring now to FIG. 12, the use of system 10 for detecting magnetic field distortion during such EM device tracking is illustrated. As previously mentioned, system 10 includes EM sensor assembly 14, tracker 16, and operator workstation 18.

In the illustrated embodiment, system 10 further includes EM transmitter 64 fixed in relation to medical (e.g., surgical) device 66 to be tracked. Device 66 may be may be any suitable device for use in a medical procedure. For example, device 66 may be a drill, a guide wire, a catheter, an endoscope, a laparoscope, a biopsy needle, an ablation device or other devices. In the illustrated embodiment, the EM transmitter 64 is mounted in the operative end of the medical device 66.

EM sensor assembly 14 includes a set of EM transmitters 34 and a set of EM receivers 36 fixed on the sensor assembly 14 with respect to each other. While the EM sensor assembly 14 of FIG. 10 with the EM transmitters 34 arranged on the periphery of the EM receivers 36 is illustrated in FIG. 12, any suitable EM sensor assembly 14 may be utilized to detect magnetic field distortions during the EM tracking procedure. During the EM tracking procedure, the EM sensor assembly 14 may be positioned in any suitable location for tracking the position of the device 66. By way of example, the EM sensor assembly 14 may fixed in relation to a patient, for example, the EM sensor assembly may be fixed in relation to a table that may be used to support a patient.

In operation, the device 66 to be tracked may be positioned within the volume of interest 12. By way of example, the device 66 may be inserted into a patient during a medical (e.g., surgical) procedure. The EM transmitter 64 mounted on the device 66 may generate an EM field. The EM receivers 36 of the EM sensor assembly 14 may measure this EM field. From these EM field measurements, the device 66 may be tracked. For example, tracker 16 may determine the position and/or orientation of the device 66. As will be appreciated, the relative positioning and functionality of the EM receivers 36 and EM transmitter 64 may be reversed. However, as those of ordinary skill in the art will appreciate, the presence of field distorting objects 28, 30 in or near the volume of interest 12 may result in distortions in the EM field generated by the EM transmitter 64 mounted in the device 66. For example, the field distorting objects may be tables, fixtures, tools, electronic equipment, one or more components of an imaging system (e.g., a C-arm). While these distortions may be compensated for using certain techniques, such as distortion maps (e.g., lookup tables that cross reference distorted and undistorted sensor position and orientation), there may be some distortion that is not compensated for. Accordingly, these field distorting objects 28, 30 generally may result in errors in the determined position and/or orientation of the device 66.

In accordance with the present technique, the EM sensor assembly 14 may be used to detect these EM field distortions, for example, that may result in errors in EM device tracking. By way of example, the EM sensor assembly 14 may be sampled to acquire EM field measurements from the EM receivers 36 of the electromagnetic fields generated by the EM transmitters 34. These EM field measurements may be monitored to detect EM field distortions within the volume of interest 12. For example, the tracker 16 may monitor the EM field measurements from the EM receivers 36 to determine an apparent location (e.g., position and/or orientation) of each of the EM transmitters 34 with respect to the EM sensor assembly 14. This determined apparent location may then be monitored to detect EM field distortions. In a similar manner, to the determined location of the device 66, the field distorting objects 28, 30 may result in distortions in the determined location of the EM transmitters 34. However, as previously mentioned, the EM transmitters 34 and the EM receivers 36 are fixed with respect to each other. As such, the location of each of the EM transmitters 34 and each of the EM receivers 36 with respect to the EM sensor assembly 14 is known. Accordingly, the determined apparent location of each of the EM transmitters 34 may be compared to this established location to detect EM field distortions within the volume of interest 12. Other aspects of the field measurements also may be monitored to detect EM field distortions, for example, the gain of a single coil and/or the mutual inductance between an EM receiver and EM transmitter. For example, the mutual inductance between one or more of the EM transmitters 34 and one or more of the EM receivers 36 may be monitored.

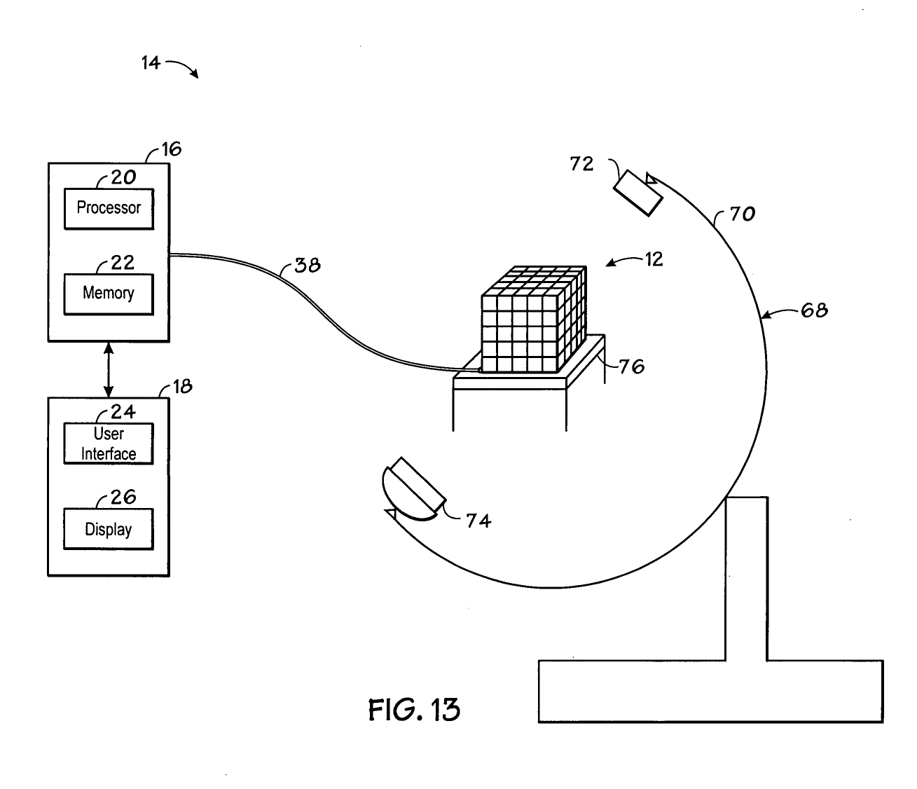

In one embodiment, the present technique may be used to characterize magnetic field distortion in a volume of interest 12, such as in the tracking volume of a clinical environment. Referring now to FIG. 13, the use of system 10 for characterizing magnetic field distortion in a clinical environment is illustrated. As previously mentioned, system 10 includes EM sensor assembly 14, tracker 16, and operator workstation 18.

In the illustrated embodiment, X-ray fluoroscopy system 68 includes a C-arm 70, an X-ray radiation source 72, and X-ray detector 74. The X-ray radiation source 72 is mounted on the C-arm 70, and the X-ray detector 74 is mounted on the C-arm 70 in an opposing location from the X-ray radiation source 72. While in some systems the X-ray radiation source 72 and the X-ray detector 74 are fixed, in a typical fluoroscopy system the C-arm 70 allows for movement of the X-ray radiation source 72 and the X-ray detector 74 about the volume of interest 12. In operation, the X-ray radiation source 72 emits a stream of radiation suitable for X-ray fluoroscopy. The X-ray detector 74 receives a portion the stream of radiation from the X-ray source 72 that passes through the volume of interest 12 in which a subject (not shown), such as a human patient, is positioned on table 76. The X-ray detector 74 produces electrical signals that represent the intensity of the radiation stream. As those of ordinary skill in the art will appreciate, these signals are suitably acquired and processed to reconstruct an image of features within the subject.

As those of ordinary skill in the art will also appreciate, the components of the X-ray fluoroscope 68, including the C-arm 70 and the table 76, will typically result in electromagnetic field distortion. Other field distorting objects may also be present. Due to this field distortion, errors in the measured sensor locations may result. In accordance with the present technique, the EM sensor assembly 14 may be used to characterize this EM field distortion, for example, so that the EM field distortion may be compensated for in subsequent EM device tracking within the volume of interest 12.

In the illustrated embodiment, the EM sensor assembly 14, shown on FIG. 13 as the box-shaped EM sensor assembly 14 from FIG. 3, is positioned on table 76. As previously mentioned, the EM sensor assembly 14 includes a set of EM transmitters 34 and a set of EM receivers 36 fixed on the EM sensor assembly 14 with respect to each other. In one embodiment, the EM sensor assembly 14 may be positioned on table 76 in the desired tracking volume to characterize EM field distortion therein. By way of example, the EM sensor assembly 14 may be sampled to obtain EM field measurements from the EM receivers 36 with respect to each of the EM transmitters 34. Based on these EM field measurements, the EM distortions within the volume of interest 12 may be characterized. In one embodiment, characterizing the distortions may include determining a location (e.g., position and/or orientation) of each of the EM receivers 36 with respect to the EM sensor assembly 14. This determined location of the EM receivers 36 may be compared to the known or established location of the EM receivers 36 with respect to the EM sensor assembly 14. As previously mentioned, because the EM transmitters 34 and EM receivers 34 are fixed with respect to each other on the EM sensor assembly 14, actual location of each of the EM sensors may be determined. By way of example, a distortion map may be created that characterizes the EM field distortion. In one embodiment, the distortion map may be in the form of a look-up table that, for example, cross-references the determined sensor locations with the established sensor location for each of the EM receivers 36 on the EM sensor assembly 14.

While specific reference is made in the present discussion to an X-ray imaging system, and particularly to a fluoroscopy system, it should be appreciated that the invention is not intended to be limited to these or to any specific type of imaging system or modality. Accordingly, the technique may be used for tracking, analysis and display of positions of implements in conjunction with other imaging modalities used in real time, or even with images acquired prior to a surgical intervention or other procedure.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method for detecting electromagnetic field distortion, comprising:
sampling a sensor assembly positioned within a volume of interest to acquire measurements of electromagnetic fields within the volume of interest, wherein the sensor assembly comprises a set of electromagnetic transmitters for generating the electromagnetic fields and a set of electromagnetic receivers for measuring the electromagnetic fields, wherein the electromagnetic transmitters and the electromagnetic receivers are disposed at fixed locations on the sensor assembly; and
monitoring the measurements to detect electromagnetic field distortion within the volume of interest.

2. The method of claim 1, wherein the monitoring the measurements comprises determining apparent locations with respect to the sensor assembly for each of the electromagnetic receivers or electromagnetic transmitters, and monitoring the determined apparent locations to detect electromagnetic field distortion within the volume of interest.

3. The method of claim 1, wherein the monitoring the measurements comprises determining apparent locations with respect to the sensor assembly for each of the electromagnetic receivers or electromagnetic transmitters, and comparing the determined apparent locations to established locations with respect to the sensor assembly for each of the corresponding electromagnetic receivers or corresponding electromagnetic transmitters.

4. The method of claim 1, wherein the monitoring the measurements comprises monitoring the mutual inductance between one or more of the electromagnetic transmitter and one or more of the electromagnetic receivers.

5. The method of claim 1, wherein the monitoring the measurements comprises monitoring gain of a single coil of one of the electromagnetic receivers or one of the electromagnetic transmitters.

6. The method of claim 1, comprising characterizing the electromagnetic field distortion based on the monitored measurements.

7. The method of claim 6, wherein characterizing the electromagnetic field distortion comprising creating a distortion map for the volume of interest based on the monitored measurements of the electromagnetic field.

8. The method of claim 6, comprising updating a determined location of a device positioned within the volume of interest based on the characterization of the electromagnetic field distortion.

9. The method of claim 1, comprising reporting a field distortion based on the monitored measurements.

10. The method of claim 1, comprising positioning a device within the volume of interest, the device comprising an electromagnetic sensor, and using the electromagnetic transmitters or electromagnetic receivers to track the device within the volume of interest.

11. The method of claim 1, wherein the measurements are monitored while a device is positioned with the volume of interest.

12. The method of claim 1, comprising monitoring mutual inductance between the electromagnetic transmitters or a calibration coil for transmitting at a known frequency and current, and the electromagnetic receivers to detect electromagnetic distortion.

13. A method for detecting electromagnetic field distortion, comprising:
positioning a sensor assembly fixed in relation to a patient, the sensor assembly comprising a set of electromagnetic transmitters and a set of electromagnetic receivers, wherein the electromagnetic transmitters and the electromagnetic receivers are disposed at fixed locations on the sensor assembly positioning a device within the patient, the device comprising an electromagnetic sensor for generating an electromagnetic field or for measuring an electromagnetic field;
tracking the position of the device with respect to the sensor assembly;
sampling the sensor assembly to obtain measurements from the set of electromagnetic receivers of electromagnetic fields generated by the set of electromagnetic transmitters; and
monitoring the measurements to detect electromagnetic field distortion within the volume of interest.

14. The method of claim 13, wherein monitoring the measurements comprises determining locations with respect to the sensor assembly for each of the electromagnetic receivers or electromagnetic transmitters, and comparing the determined locations to established locations for each of the electromagnetic receivers or electromagnetic transmitters with respect to the sensor assembly.

15. The method of claim 13, wherein the monitoring the measurements comprises monitoring the mutual inductance between one or more of the electromagnetic transmitter and one or more of the electromagnetic receivers.

16. The method of claim 13, wherein the monitoring the measurements comprises monitoring gain of a single coil of one of the electromagnetic receivers or one of the electromagnetic transmitters.

17. A system for detecting electromagnetic field distortions, comprising:
a sensor assembly for positioning within a volume of interest, the electromagnetic sensor assembly comprising a set of electromagnetic receivers, and a set of electromagnetic transmitters, wherein the electromagnetic receivers and the electromagnetic transmitters are disposed at fixed locations on the sensor assembly; and
a tracker configured to sample the sensor assembly to acquire measurements of electromagnetic fields generated by the electromagnetic transmitters, and monitor the measurements to detect electromagnetic field distortion within the volume of interest.

18. The system of claim 17, wherein to monitor the measurements the tracker is configured to determine locations with respect to the sensor assembly for each of the electromagnetic receivers or electromagnetic transmitters, and monitor the determined locations to detect electromagnetic field distortion within the volume of interest.

19. The system of claim 17, wherein to monitor the measurements the tracker is configured to determine locations with respect to the sensor assembly for each of the electromagnetic receivers or electromagnetic transmitters, and compare the determined locations to established locations with respect to the sensor assembly for each of the electromagnetic receivers or electromagnetic transmitters.

20. The system of claim 17, wherein to monitor the measurements the tracker is configured to monitor the mutual inductance between one or more of the electromagnetic transmitters and one or more of the electromagnetic receivers.

21. The system of claim 17, wherein to monitor the measurements the tracker is configured to monitor gain of a single coil of one of the electromagnetic receivers or one of the electromagnetic transmitters.

22. The system of claim 17, wherein the sensor assembly comprises a sensor panel, wherein the set of electromagnetic transmitters and the set of electromagnetic receivers are mounted on the sensor panel.

23. The system of claim 22, wherein the electromagnetic transmitters and the electromagnetic receivers are mounted on the sensor panel in a series of rows such that each row alternates between one of the electromagnetic transmitters and one of the electromagnetic receivers.

24. The system of claim 17, wherein the sensor assembly comprises a plurality of sensor panels arranged in two or more planes, wherein each of the sensor panels comprises one or more of the set of electromagnetic receivers and one or more of the set of electromagnetic receivers.

25. The system of claim of claim 24, wherein the sensor assembly comprises a box, wherein the box comprises one of the plurality of sensor panels on two or more sides of the box.

26. The system of claim 25, wherein the box has an open bottom, and wherein the sensor assembly is configured to be placed around a head of a patient.

27. The system of claim 25, wherein the box has an open top and an open bottom, and wherein the sensor assembly is configured to placed around a torso of a patient.

28. The system of claim 17, wherein the sensor assembly comprises a plurality of sensor panels arranged in two or more planes, wherein one of the electromagnetic transmitters or one of the electromagnetic receivers is generally positioned in the center of each sensor panel, and wherein one of the electromagnetic transmitters or one of the electromagnetic receivers is generally positioned in each corner of each sensor panel.

29. The system of claim 17, wherein the sensor assembly comprises a rack system and a plurality of sensor panels mounted in the rack system, wherein each of the sensor panels comprises one or more of the electromagnetic transmitters and one or more of the electromagnetic receivers.

30. The system of claim 17, wherein the set of electromagnetic transmitters are arranged on the sensor assembly on the periphery of the set of electromagnetic receivers.

31. The system of claim 30, wherein the sensor assembly comprises a printed circuit board, wherein the printed circuit board comprises the set of electromagnetic receivers printed thereon.

32. The system of claim 31, wherein each of the electromagnetic receivers printed on the printed circuit board comprise a single coil, and wherein each of the electromagnetic transmitters arranged on the periphery of the electromagnetic receivers comprise three dipole coils.

33. The system of claim 31, wherein the printed circuit board comprises a calibration coil printed thereon, wherein the calibration coil is configured to transmit at a known frequency and current.

34. The system of claim 30, wherein the set of electromagnetic transmitters are on a different plane than the set of electromagnetic receivers.

35. The system of claim 30, wherein the sensor assembly comprises a calibration coil fixed on the sensor assembly, wherein the calibration coil is configured to transmit at a known frequency and current.
